(19) United States
(12) Patent Application Publication
(10) Pub. No.: US 2014/0171782 A1
(43) Pub. Date: Jun. 19, 2014

(54) METHOD FOR DETECTING THE POSITION OF A TRANSDUCER

(71) Applicants: Ralf Bruder, Luebeck (DE); Gerd Bruder, Luebeck (DE); Achim Schweikard, Hamburg (DE)

(72) Inventors: Ralf Bruder, Luebeck (DE); Gerd Bruder, Luebeck (DE); Achim Schweikard, Hamburg (DE)

(73) Assignee: UNIVERSITAET ZU LUEBECK, Luebeck (DE)

(21) Appl. No.: 14/236,184

(22) PCT Filed: Jul. 27, 2012
(86) PCT No.: PCT/DE2012/100228

§ 371 Date: Jan. 30, 2014

(30) Foreign Application Priority Data
Jul. 30, 2011 (DE) 10 2011 109 037.5

(51) Int. Cl. A61B 8/00; A61B 5/00

(52) U.S. Cl. CPC A61B 8/429; A61B 5/0035
USPC 600/411; 600/427

(57) ABSTRACT

A method for detecting the position of a transducer for monitoring the position and motion of one or more target structures for the preparation or during an operation, with creating at least one volume data set (CT or MRI) showing the target structure(s), possible contact surfaces for the positioning of the ultrasonic transducer and the tissue between contact surfaces and target structure(s), determining from the volume data set one or more contact surfaces on which the best reflection of the ultrasound is or are to be expected, and positioning the ultrasonic transducer which monitors the operation on the contact surface(s).

### BACKGROUND OF THE INVENTION

Radiotherapy is a proven means for treating tumor tissue. Focused ionizing radiation is directed from different directions from the outside of the human body onto the tumor. Since the effect is achieved in the target area by a cumulative dose of radiation, multiple radiation beams may be weighted from different spatial angles in order to protect the surrounding tissue and, in particular, to unburden critical structures. The CyberKnife (Accuray Inc.) and the Trilogy (Varian Medical Systems) system are two robotic systems for radiation therapy.

Modern radiation therapy systems include supplemental imaging systems to verify target positions and to treat tumors that are subject to respiratory motion. There are also efforts to treat target structures in the region of the heart. An example is the treatment of atrial fibrillation, wherein uncoordinated electrical stimuli greatly reduced the pumping capacity of the atria and trigger cardiac fibrillation. Parallel to invasive catheter ablation, this involves an attempt to generate radiation scar tissue in the heart and in this way to suppress roaming electrical pulses.

The speed of movement of target structures in the area of the heart can be significantly higher than the speed of lung tumors under respiration. Moreover, since several critical structures lie in the immediate vicinity of the target area and since an accurate patient-alignment is necessary, an image-based monitoring of the target area and motion compensation with a high sampling rate is recommended during the entire procedure.

Ultrasound imaging represents, for both cardiovascular and for conventional radiation surgery, a rapid, non- ionizing alternative to existing x-ray imaging. It has been shown that the motion information of targets in ultrasound images can be extracted (for example, by pattern matching). This information can be used in different ways for motion compensation. The target structure can be located directly in the ultrasound image and the radiation source aligned with this target, or can be continuously followed. An alternative is to use the correlation between low-frequency sampled absolute position of the target structure (located by stereo X-ray images of gold markers in the target area) for fast location tracking in the ultrasound image. In this way, a current high resolution target position can be calculated from the ultrasound location and used for repositioning the radiation beam. The basis for this method is to have the most accurate localization of the target movement in the ultrasound image.

For motion detection, ultrasound systems adapted to the area of study can be used. To visualize the heart, for example, selection could be made from available transthoraxiale (TTE) or transesophageal (TEE) probes. The data (here called continuous ultrasound images), can be detected in one, two or three dimensions and be used for the extraction of position information. During a procedure the probes can be static, robot carried or can be fixed at a selected transducer position by adhering to the skin.

The emitted ultrasound penetrates from this position the tissue to be displayed and thereby changes—depending on the characteristics of the penetrated tissue—it's energy and speed. This has the following problems:

A possible consequence of these operations is that not enough energy reaches deeper layers for imaging this. Air inclusions and bone reflect or absorb a large part of the sound and impede the appearance of underlying tissue layers. Especially in the area of the heart, which is obscured by the lung lobes and the rib cage, the search for a suitable transducer position to visualize a particular target structure is difficult. In addition, large parts of the upper body undergo a combination of voluntary and involuntary movements (respiration, pulsation). Depending on the type and duration of monitored therapy, a visualization must be ensured over the entire treatment period.

For reliable position location in the ultrasound image, two other problems arise:

For an automatic localization and tracking of a target structure, this must have an ultrasound image of sufficient intensity. If in this area little reflection takes place, or if the ultrasound is reflected at an angle other than back to the transducer, the representation of the target region for an object tracking may be insufficient.

A tissue sonic impulse travel time or run time deviating from the average sonic impulse travel time in the human body will have the consequence that distances in the ultrasound image will be reproduced with error. This error is up to seven percent of the distance between the transducer and the target structure. For the distance between the transducer and the target structure there applies, depending on time t

d_{measured}(t)=d_{Real}(t)+d_{measurement error}(t)

with the location error dependent on the distance between the transducer and the target structure d_{Real} and the quotient of the standard sonic impulse travel time assumed for the ultrasonic unit □_{standard} and the real sonic impulse travel time in the tissue □ᵣₑₐₗ

$${d}_{measurement error} ( t ) = {d}_{Real} ( t ) \cdot \frac{{v}_{Real} ( t )}{{v}_{Standard}}$$

For the stationary case, outside of the target area □ᵣₑₐₗ(t)=c is constant. The relative proper movement of the target structure exhibits in this case a small error and for small proper movement can be approximated by

Δd_{measured}(t)=Δd_{Real}(t)+Δd_{measurement error}(t)≈Δd_{Real}(t)

To use the absolute position of the target structure in the ultrasound image the distance errors must are calculated, which is possible in various ways by calibrating the value c, such as by position-referenced average values, simulation results or additional localization of known static structures with known transducer distance in the ultrasound image and the comparison of measured and known distance information.

If however the tissue between the target structure and the transducer is itself subjected to a movement, and if this results in a change in the sonic impulse travel time between target structure and transducer, then the relative change of the resulting run-time error can, as a function of the distance dᵣₑₐₗ, may be of a similar order of magnitude as the proper motion of the target structure.

Δd_{measured}(t)=Δd_{Real}(t)+Δd_{measurement error}(t)+d_{measurement error}(t)

Since voluntary and involuntary movements overlap in the thoracic region, an estimate of the error occurring is extremely difficult. Both the absolute position information of the target structure in the ultrasonic image, as well as the relative movement information, are not utilizable for motion compensation.

### STATE OF THE ART

In HIFU (high intensity focused ultrasound) in a current research project fabric properties are mapped to supersonic speeds in order to achieve a clean as possible superposition of all incoming ultrasonic energy into a sharp focal point. But the goal is the destruction of tissue by ultrasound and not object location.

In echocardiography, there are standard positions for recording ultrasound images of the heart (so-called “acoustic windows”), which allow an unobstructed view of the heart in certain patient positions and with held breath. Thus the problem of visibility is at least partially overcome. In radiation therapy, however, a patient must forcibly lie on his back. A therapy session lasts up to 30 minutes, making breath-holding difficult. The targets are tumors or structures on the heart, which often lie outside the standard views.

### THE OBJECT OF THE INVENTION

The invention is thus concerned with the objective to provide a method for unobstructed location of one or more target structures in the ultrasound image. Therewith, for a given probe position, the imaging must be made possible
the target structure(s) in a defined state
the moving target structure(s) (due to respiration and pulsation (omit?)
the moving target structure(s) due to simultaneous movement of the surrounding structures or the structure lying between target and transducer.

Optionally, the measured target structure movement information is to be as free as possible of measurement errors occurring due to tissue motion between the transducer and the target area.

### SOLUTION OF THE PROBLEM

According to the invention this object is achieved by the features of claim 1. The dependent claims describe preferred embodiments of the invention.

According to the invention it is proposed, prior to the procedure, to define a (preferably several) planning volume (CT or MRI) of the area to be imaged between the possible positions of the transducer and the target structure. Then, the ultrasonic acoustic impedance and ultrasound (ultrasonic impulse) travel times are classified from the planning images. The optimum position of the ultrasound transducer is then calculated by evaluating every possible transducer position based on the determined variables, and the ultrasonic transducer head of the monitoring ultrasonic system is then positioned accordingly.

The invention relates to a method for detecting the position of a transducer for monitoring the position and motion of one or more target structures for the preparation or during a procedure, with creating at least one volume data set (CT or MRI), showing the target structure(s), possible contact surfaces for the positioning of the ultrasonic transducer and the tissue between contact surfaces and target structure(s), determining from the volume data set one or more contact surfaces on which the best reflection of the ultrasound is or are to be expected, and positioning the ultrasonic transducer which monitors the intervention on the contact surface(s).

The tissue represented in the planning volume is assigned its acoustic properties (sound velocity, acoustic impedance). The assignment can be, for example, based on the spatial position (classification of segmented regions) or by use of an appropriate transfer function between intensity values in the planning volume and acoustic properties.

On the basis of these properties by every possible transducer position from the view of the target structure(s) is simulated. This can be done in different ways: complex ultrasound simulators (see U.S. Pat. No. 7,835,892, U.S. Pat. No. 7,731,499 and U.S. Pat. No. 7,699,778) create a virtual ultrasound image of the planning volume. As visibility or image quality criterion of the target structure, the brightness (reflection) or the entropy can be evaluated in the target area in the generated ultrasound image. Alternatively, the tissue absorption for the sound propagating in the direct line of sight between the transducer and the target structure and the reflection of the sound in the target area can be used for the approximation of the visibility of the target structure. In addition to the visibility of the target structure, the sound travel time between target structure(s) and the transducer is simulated based on the data in the planning volume.

If the expected location area of a target structure is determined to be in a planning volume, then visibility (line of sight) and sound travel time for each transducer position are simulated for each possible target position in the occupied zone. If several volume data sets for different states of motion of the target structure and surrounding tissue are available, then the calculation is performed in parallel on all planning data sets.

To minimize the measurement error due to tissue motion, the simulated sound travel times are analyzed in view of the available planning data sets and the measurement task. Criteria are
the expected deviation of sound travel time from the standard sound travel time assumed by the ultrasound device,
the change of the sound travel time to a target structure for the anticipated target motion in
the planning volume,
the change of the sound travel time to a target structure for multiple planning volumes,
the difference in sound travel times to multiple target structures in the ultrasound image.

An algorithm selects, depending upon the given visibilities and acoustic criteria, one or more transducer positions. (The transducer is placed on this position).

One example is the use of the method for positioning an ultrasound transducer for motion compensation in a robotic, image-guided radiotherapy (IGRT). The task is to seamless or uninterrupted tracking of a structure (tumor, treatment area) in the area of the human thorax, where a respiratory and/or pulsating movement may be present.

In preparation for the treatment step usually one or more CT planning volumes are created that depict the thorax in various respiratory conditions or heart phases. On the basis thereof the radiosurgical intervention can be planned by segmentation of target and risk structures and optimization of the weighting of a multiple of possible sets of rays from different directions onto the target area.

The method described here is implemented in this pre-processing step. Based on the CT volume data possible contact surfaces for application of the transducer are determined, for example by extraction of the skin surface. The various positions are then subjected to an evaluation, as to what extent they are suitable for the observation of a target structure inside the thorax by ultrasound.

For this, first the visibility of a target structure used for motion detection is checked.

TABLE 1
Assignment of Hounsfield values to sound properties

| | | Sound- | Sound- |
|---|---|---|---|
| | Hounsfield-Units | Impedance | Velocity |
| Tissue | (min/max) | (kg(m²/s) | (m/s) |
| Pure Air | −1000 | 0.0004 | 331 |
| Lung | −800/−500 | 0.003 | 331 |
| Fatty Tissue | −100/10 | 0.138 | 1468 |
| Watter | −10/10 | 1.53 | 1526 |
| Liver | 40/60 | 1.65 | 1559 |
| Bone | 250/1000 | 6.66 | 3600 |
| Blood | 30/70 | 1.60 | 1562 |
| Cardiac Tissue | 20/50 | 1.67 | 1590 |

Table 1 gives an overview of the tissue in the region of the heart with the therewith associated typical intervals of the CT measurable Hounsfield units. The different materials are compared against their average acoustic properties (acoustic impedance, sound velocity, etc.). Using these data, the acoustic properties of the anatomy are associated with or assigned to the voxels of the planning volume.

The evaluation of the target visibility occurs in the framework of a simplified model for sound absorption in the tissue, in which the planning volume from the ultrasonic head to the target structure is run through in a direct connecting line, while the absorption of the emitted sonic pulse is calculated. In simplified manner, reflection and scattering can be calculated and integrated as the main portions of the absorption from the Hounsfield units of the volume voxels lying in the path. Other factors—generally affecting the absorption of the beam—such as interference and refraction are ignored in this model.

In this way the target visibility is defined for all possible positions of the ultrasound transducer via all planning volumes as the absorption-diminished percentage of the target structure reaching the ultrasound transducer.

If several target positions or planning volumes exist for the respective transducer position, the target visibility for the transducer position is calculated as the minimum of the individual target visibilities.

An optimal probe position can be found by optimizing the target visibility over all transducer positions. Furthermore, a threshold for acceptable visibility can be used and all transducer positions with visibilities above this threshold value can be used for further processing.

One of these processing steps is to minimize tissue motion induced time- and position-dependent distance error between the transducer and the target structure in the position measurement of the target structure. For this purpose, in a second optimization step, among all transducer positions with sufficient target visibility, the position with the lowest expected distance error is selected. Parallel to the determination of the absorption, the sound propagation time is determined on the direct connecting line between the transducer and the target. Depending on the measuring task there arise the following optimization tasks:
For the absolute position measurement of target structures, the difference between the standard (default) speed of sound and the speed calculated from the tissue properties, the real sound travel time, must be minimized. As the error function, there can be used here the RMS error of the speed differences on the direct line between the transducer and the target structure. A minimization of this function provides the optimal transducer position.
If a relative motion information of the target structure is to be obtained, such as for correlation, the change in the sound travel time between the transducer and the target structure must be minimized. Across all target positions and planning volumes the RMS error between the calculated sound travel time and the average, calculated sound transit time is defined as the error function and is minimized.

Subsequently, the ultrasonic transducer head is placed onto the calculated position.

The well-known the prior art methods differ by from the present invention substantially by:
the use of a focused ultrasound as a therapy tool and
the continuous monitoring during the surgery with MR and
the use of an ultrasound array.

The present invention, however, is used for imaging, whereas MRI or CT are used for planning before the procedure. And in particular the use of only one ultrasound transducer head is to be noticed as a special feature.

1. A method for finding the position of a transducer for monitoring the position and motion of one or more target structures for preparation prior to, or during, an operation, comprising
parallel simulation of virtual ultrasound images from a plurality of volume data sets (CT/MRI) for different states of motion of target structures and surrounding tissue for a preselected transducer position on a possible contact area
determining the target visibility as the minimum of the absorption-attenuated proportion of the ultrasound reaching the target structure for all simulated ultrasound images,
varying the ultrasonic transducer head position on the contact surface, and
positioning on the contact surface with the largest target visibility.

2. The method according to claim 1, comprising
associating ultrasonic properties such as speed of sound and acoustic impedance to structures from the volume data set by a local function of the intensity values in the volume data set or the segmentation of different acoustic properties in the volume data set and assigning the sound characteristics to the segmented regions, and
determining one or more contact surfaces from among all possible contact surfaces, at which the reflection(s) and absorption(s) of the underlying tissue between target structure the transducer allow the introduction of the highest sound intensity (sonic pulse), or the minimum sound intensity in the target structure, (and therewith a minimum of image quality in the ultrasound imaging).

3. The method according to claim 2, comprising
calculating the optimal contact surface considering movement of the target structure, or the structures located upstream of the target structure, the sound intensity at a contact surface from the minimum of the individual sound intensities is calculated in a plurality of volume data sets and (or) for multiple positions of the target structure.

4. The method according to claim 2, comprising
selecting the optimum transducer position from the calculated potential contact surfaces with minimum sound intensity, at which the sound propagation times of the upstream structures between contact area and target structures changes as little as possible over time.

5. The method according to claim 2, comprising
selecting an optimal transducer position from the calculated potential contact surfaces with minimum sound intensity, at which the sound propagation times between the contact surface and the individual target structures differ from each other as little as possible.
